(19) United States
(12) Patent Application Publication
(10) Pub. No.: US 2021/0000411 A1
(43) Pub. Date: Jan. 7, 2021

(54) COGNITIVE FUNCTION EVALUATION DEVICE, COGNITIVE FUNCTION EVALUATION SYSTEM, COGNITIVE FUNCTION EVALUATION METHOD, AND RECORDING MEDIUM

(71) Applicant: Panasonic Intellectual Property Management Co., Ltd., Osaka (JP)

(72) Inventors: Ryosuke NAGUMO, Osaka (JP); Sadayuki SUMI, Hyogo (JP); Mitsuharu HOSOKAWA, Osaka (JP); Kengo ABE, Nara (JP); Yoshihiro MATSUMURA, Osaka (JP); Takashi NISHIYAMA, Hyogo (JP); Makoto KARIYASU, Kyoto (JP); Takako YOSHIMURA, Hyogo (JP); Minoru TOYAMA, Oita (JP)

(21) Appl. No.: 16/978,313

(22) PCT Filed: Mar. 15, 2019
(86) PCT No.: PCT/JP2019/010799

§ 371 Date: Sep. 4, 2020

(30) Foreign Application Priority Data
Mar. 29, 2018 (JP) 2018-065949

(51) Int. Cl. A61B 5/00; G10L 25/90; G10L 25/66

(52) U.S. Cl. CPC A61B 5/4803; A61B 5/4088; G10L 25/66; G10L 25/90

(57) ABSTRACT

A cognitive function evaluation device includes an acquirer that acquires speech data that indicates speech uttered by an evaluatee in response to a task given to the evaluatee; an evaluator that evaluates a cognitive function of the evaluatee by performing a first evaluation based on a proportion of speaking time of the evaluatee in a predetermined period in the speech data acquired by the acquirer and a second evaluation based on a fundamental frequency in the speech data acquired by the acquirer; and an outputter that outputs an evaluation result obtained from the evaluation performed by the evaluator.

### TECHNICAL FIELD

The present invention relates to a cognitive function evaluation device, a cognitive function evaluation system, a cognitive function evaluation method, and a program, with which the cognitive function of an evaluatee can be evaluated.

### BACKGROUND ART

Conventionally, methods in which an evaluatee who is a patient whose cognitive function is evaluated fills a questionnaire, such as revised Hasegawa's dementia scale (HDS-R), MMSE (Mini-Mental State Examination), and CDR (Clinical Dementia Rating), are known as tests for evaluating cognitive function. These tests are conducted for evaluatees in medical facilities by doctors, clinical psychologists, and the like who have received specialized training.

The evaluation methods that use questionnaires are problematic in that it requires a long test time, which imposes a burden on the evaluatee. In addition, there is another problem in that, when repeating the same test for the same evaluatee, the evaluatee may remember the questions because the same test is used. In order to solve this problem, a technique is disclosed in which questions and answers between a doctor or the like and an evaluatee are recorded, and the speech of the evaluatee is analyzed (see, for example, Patent Literature 1).

### CITATION LIST

### Patent Literature

PTL 1: WO 2012/165602

### SUMMARY OF THE INVENTION

### Technical Problem

There is a need for a cognitive function evaluation, with which the cognitive function of an evaluatee can be more easily evaluated.

Accordingly, it is an object of the present invention to provide a cognitive function evaluation device and the like, with which the cognitive function of an evaluatee can be easily evaluated.

### Solutions to Problem

A cognitive function evaluation device according to an aspect of the present invention includes: an acquirer that acquires speech data that indicates speech uttered by an evaluatee in response to a task given to the evaluatee; an evaluator that evaluates a cognitive function of the evaluatee by performing a first evaluation based on a proportion of speaking time of the evaluatee in a predetermined period in the speech data acquired by the acquirer and a second evaluation based on a fundamental frequency in the speech data acquired by the acquirer; and an outputter that outputs an evaluation result obtained from the evaluation performed by the evaluator.

A cognitive function evaluation system according to an aspect of the present invention includes: the above-described cognitive function evaluation device; a sound collecting device that detects speech uttered by the evaluatee; and a display device that displays an evaluation result output by the outputter.

A cognitive function evaluation method according to an aspect of the present invention is a cognitive function evaluation method executed by a computer, the cognitive function evaluation method including: acquiring speech data that indicates speech uttered by an evaluatee in response to a task given to the evaluatee; evaluating a cognitive function of the evaluatee by performing a first evaluation based on a proportion of speaking time of the evaluatee in a predetermined period in the speech data acquired in the acquiring of the speech data and a second evaluation based on a fundamental frequency in the speech data acquired in the acquiring of the speech data; and outputting an evaluation result obtained from the evaluation in the evaluating of the cognitive function of the evaluatee.

A program according to an aspect of the present invention is a program that causes a computer to execute the above-described cognitive function evaluation method.

### Advantageous Effect of Invention

With the cognitive function evaluation device and the like of the present invention, the cognitive function of an evaluatee can be easily evaluated.

### BRIEF DESCRIPTION OF DRAWINGS

FIG. 1 is a diagram showing a configuration of a cognitive function evaluation system according to an embodiment.

FIG. 2 is a block diagram showing a characteristic functional configuration of a cognitive function evaluation device according to the embodiment.

FIG. 3 is a flowchart illustrating a processing procedure for evaluating the cognitive function of an evaluatee, performed by the cognitive function evaluation device according to the embodiment.

FIG. 4 is a diagram showing an example of an image indicating an evaluation result.

FIG. 5 is a diagram showing a first example of a method for acquiring speech data.

FIG. 6 is a diagram showing a second example of a method for acquiring speech data.

FIG. 7 is a diagram showing a third example of a method for acquiring speech data.

FIG. 8 is a diagram showing a fourth example of a method for acquiring speech data.

FIG. 9 is a diagram showing a fifth example of a method for acquiring speech data.

FIG. 10 is a diagram illustrating the proportion of speaking time.

FIG. 11 is a diagram showing variations in fundamental frequency with time.

FIG. 12 is a diagram showing a configuration of a cognitive function evaluation system according to Variation 1 of the embodiment.

FIG. 13 is a diagram showing a configuration of a cognitive function evaluation system according to Variation 2 of the embodiment.

### DESCRIPTION OF EXEMPLARY EMBODIMENT

Hereinafter, an embodiment will be described with reference to the drawings. The embodiment described below shows a generic or specific example. The numerical values, shapes, materials, structural elements, the arrangement and connection of the structural elements, steps, the order of the steps, and the like shown in the following embodiment are merely examples, and therefore are not intended to limit the scope of the present invention. Also, among the structural elements described in the following embodiment, structural elements not recited in any one of the independent claims are described as arbitrary structural elements.

In addition, the diagrams are schematic representations, and thus are not necessarily true to scale. Also, in the diagrams, structural elements that are substantially the same are given the same reference numerals, and a redundant description may be omitted or simplified.

Also, in the embodiment given below, terms indicating directions are used. For example, the term “parallel” means not only being completely parallel, but also being substantially parallel, which means, for example, a deviation of about several percent may be included.

### Embodiment

### [Configuration of Cognitive Function Evaluation Device]

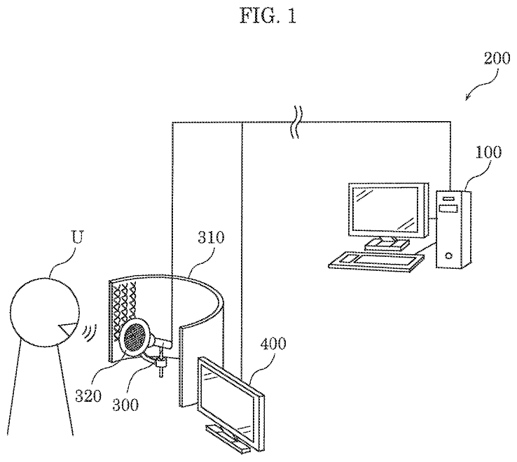

A configuration of a cognitive function evaluation system according to an embodiment will be described. FIG. 1 is a diagram showing a configuration of a cognitive function evaluation system according to an embodiment.

Cognitive function evaluation system 200 is a system for evaluating the cognitive function of evaluatee U based on the speech of evaluatee U. As used herein, the term “cognitive function” refers to the abilities of recognition, remembering information, making decisions, and the like. Specifically, for example, cognitive function evaluation device 100 performs evaluation as to whether or not the evaluatee is a person with dementia (dementia patient).

As used herein, the term “dementia” refers to symptoms of impairment of the cognitive function. A specific example of dementia is Alzheimer's disease (AD). A person with dementia cannot notice any symptoms of dementia by himself/herself, and thus the dementia patient is convinced and taken to a doctor for a medical examination by a family member or an acquaintance of the dementia patient. Also, whether or not evaluatee U has dementia can be checked by taking a batch test for diagnosing dementia such as MoCA (Montreal Cognitive Evaluation) test.

However, the MoCA test takes about 15 minutes. In addition, in order to diagnose chronological variations of evaluatee U, the MoCA test needs to be performed a plurality of times every predetermined day to determine whether or not evaluatee U has dementia. That is, the MoCA test requires a long time to diagnose whether or not evaluatee U has dementia.

It is known that even if a dementia patient and a person who is not suffering from dementia (a cognitively healthy person) utter the same word, there are differences in their voices.

Cognitive function evaluation system 200 is a system that accurately evaluates the cognitive function of evaluatee U by analyzing the speech of evaluatee U.

As shown in FIG. 1, cognitive function evaluation system 200 includes cognitive function evaluation device 100, sound collecting device 300, and display device 400.

Cognitive function evaluation device 100 is a computer that acquires speech data that indicates speech uttered by evaluatee U by using sound collecting device 300, and evaluates the cognitive function of evaluatee U based on the acquired speech data.

Sound collecting device 300 is a microphone that detects speech uttered by evaluatee U and outputs speech data that indicates the detected speech to cognitive function evaluation device 100. In order to accurately detect the speech uttered by evaluatee U, sound-insulating wall 310 and/or pop filter 320 may be provided around sound collecting device 300.

Display device 400 displays an image based on image data output from cognitive function evaluation device 100. Display device 400 is specifically a monitor device composed of a liquid crystal panel, an organic EL panel, or the like. Alternatively, a television set, a smartphone, or an information terminal such as a tablet terminal may be used as display device 400.

It is sufficient that cognitive function evaluation device 100, sound collecting device 300, and display device 400 are connected such that they can transmit and receive sound data and image data, and they may be connected with a cable, or may be connected so as to be capable of wireless communication.

Cognitive function evaluation device 100 analyzes the speech of evaluatee U based on the speech data detected by sound collecting device 300, evaluates the cognitive function of evaluatee U based on the result of analysis, and outputs, to display device 400, image data for displaying an image that indicates the evaluation result. By doing so, cognitive function evaluation device 100 can inform a dementia patient who has not noticed any symptoms of dementia of the level of cognitive function, and it is therefore possible to, for example, advise the dementia patient to consult a doctor for a medical examination. In other words, cognitive function evaluation device 100 can help a dementia patient who has not noticed any symptoms of dementia consult a doctor and take a medical examination by informing the dementia patient of the level of cognitive function.

Cognitive function evaluation device 100 is, for example, a personal computer, but may be a server device.

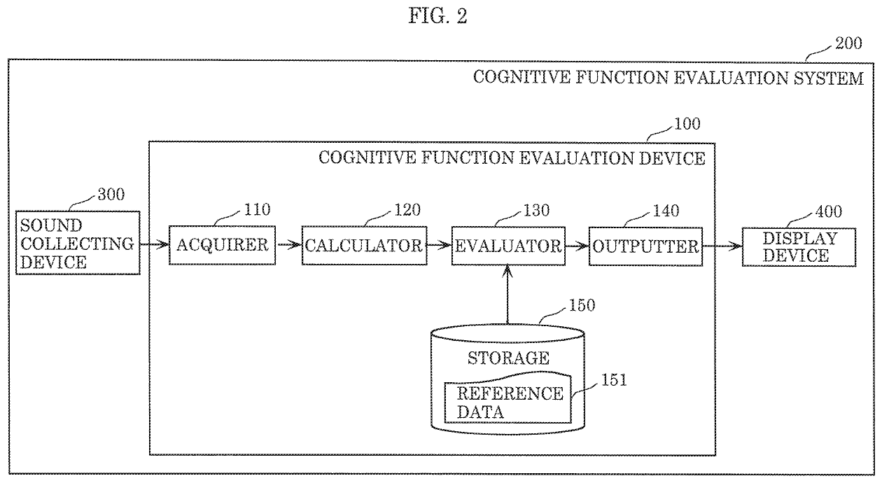

FIG. 2 is a block diagram showing a functional configuration of cognitive function evaluation device 100. Cognitive function evaluation device 100 includes acquirer 110, calculator 120, evaluator 130, outputter 140, and storage 150.

Acquirer 110 acquires the speech data detected by sound collecting device 300. Acquirer 110 is, for example, a communication interface for performing wired communication or wireless communication.

Calculator 120 is a processing unit that analyzes the speech data of evaluatee U acquired by acquirer 110. Calculator 120 is specifically implemented by a processor, a microcomputer, or a dedicated circuit.

Evaluator 130 evaluates the cognitive function of evaluatee U by comparing the result of speech data analysis performed by calculator 120 with reference data 151 stored in storage 150. Evaluator 130 is specifically implemented by a processor, a microcomputer, or a dedicated circuit.

Outputter 140 outputs, to display device 400, the evaluation result obtained as a result of evaluator 130 evaluating the cognitive function of evaluatee U. Outputter 140 is, for example, a communication interface for performing wired communication or wireless communication.

Storage 150 is a storage device in which reference data 151 that is used as the basis for evaluating the cognitive function of a person is stored. Reference data 151 is referenced by evaluator 130 when evaluating the level of cognitive function of evaluatee U. Storage 150 is implemented by, for example, a ROM (Read Only Memory), a RAM (Random Access Memory), a semiconductor memory, a HDD (Hard Disk Drive), or the like.

In storage 150, programs executed by calculator 120 and evaluator 130 and image data that indicates evaluation results of evaluation of the cognitive function of evaluatee U used when outputting evaluation results are also stored.

### [Processing Procedure of Cognitive Function Evaluation Method]

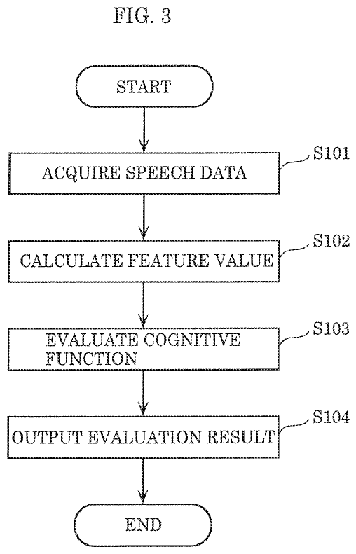

Next, a specific processing procedure of a cognitive function evaluation method executed by cognitive function evaluation device 100 will be described. FIG. 3 is a flowchart illustrating a processing procedure for evaluating the cognitive function of evaluatee U, performed by cognitive function evaluation device 100.

First, acquirer 110 acquires the speech data of evaluatee U via sound collecting device 300 (step S101).

Next, calculator 120 calculates the feature value from the speech data acquired by acquirer 110 (step S102). In step S102, for example, calculator 120 calculates the proportion of speaking time of evaluatee U in a predetermined period within the speech data as the feature value. Alternatively, calculator 120 calculates the fundamental frequency in the speech data as the feature value.

Next, evaluator 130 evaluates the cognitive function of evaluatee U based on the feature value calculated by calculator 120 in step S102 (step S103). In step S103, reference data 151 stored in storage 150 is used as the basis for evaluation.

Next, outputter 140 outputs an evaluation result obtained as a result of evaluator 130 evaluating the cognitive function of evaluatee U (step S104). In step S104, for example, outputter 140 acquires, from storage 150, image data of an image indicating the evaluation result obtained as a result of evaluator 130 performing evaluation in step S103, and transmits the acquired image data to display device 400.

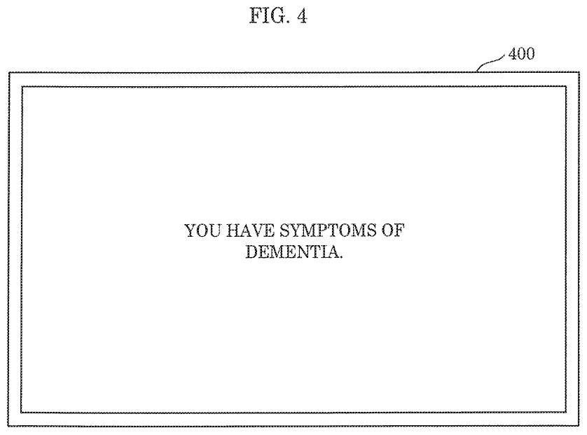

Display device 400 acquires the image data output by outputter 140, and displays an image based on the image data. FIG. 4 is a diagram showing an example of an image indicating an evaluation result. The image shown in FIG. 4 indicates that evaluatee U has symptoms of cognitive function impairment.

In this way, as a result of the evaluation result being displayed as an image, evaluatee U can easily see the result of cognitive function evaluation. In the case where cognitive function evaluation is performed by evaluatee U at home or the like by using cognitive function evaluation device 100, cognitive function evaluation device 100 can advise evaluatee U to consult a doctor or the like by displaying an image.

### [Method for Acquiring Speech Data]

Next, the method for acquiring speech data performed in step S101 will be described. In step S101, in order for the level of dementia of evaluatee U to be significantly noticeable in speech data, speech data is acquired in a situation where a certain level of stress is applied to the brain of evaluatee U. Specifically, a task is given to evaluatee U, and acquirer 110 acquires speech data that indicates speech uttered by evaluatee U in response to the task given to evaluatee U.

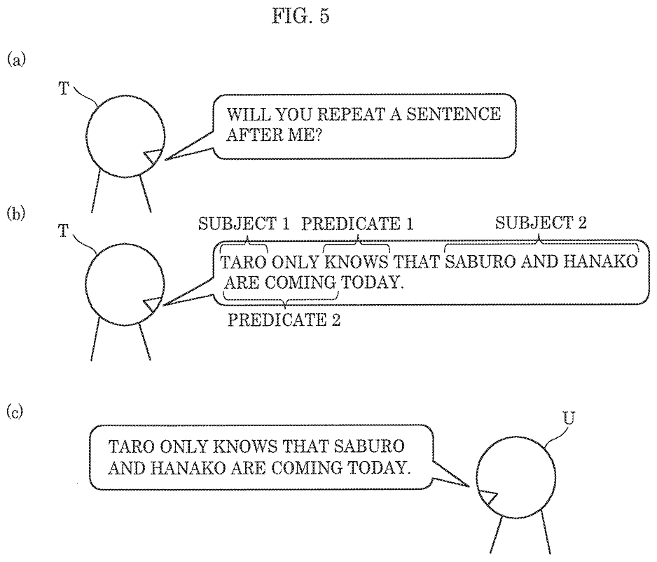

There are various methods of giving a task. Hereinafter, examples of methods for acquiring speech data will be described. FIG. 5 is a diagram showing a first example of a method for acquiring speech data.

In the example of FIG. 5, evaluatee U is given a task to verbally repeat a sentence spoken by tester T. Tester T first gives an instruction to verbally repeat a sentence to evaluatee U ((a) in FIG. 5), and then speaks a sentence ((b) in FIG. 5). Then, evaluatee U speaks in response to the task ((c) in FIG. 5). Acquirer 110 acquires speech data of the speech uttered by evaluatee U.

There is no particular limitation on the sentence used. However, as described above, for the purpose of applying a certain level of stress to the brain of evaluatee U, a slightly complex sentence may be used. For example, in (b) in FIG. 5, a sentence that contains a plurality of pairs of a subject and a predicate is used. Also, in order to acquire speech data with a certain length, for example, a sentence that contains 5 phrases or more is used. Also, instead of the task to verbally repeat a sentence, a task to verbally repeat a plurality of numbers may be used as the task given to evaluatee U.

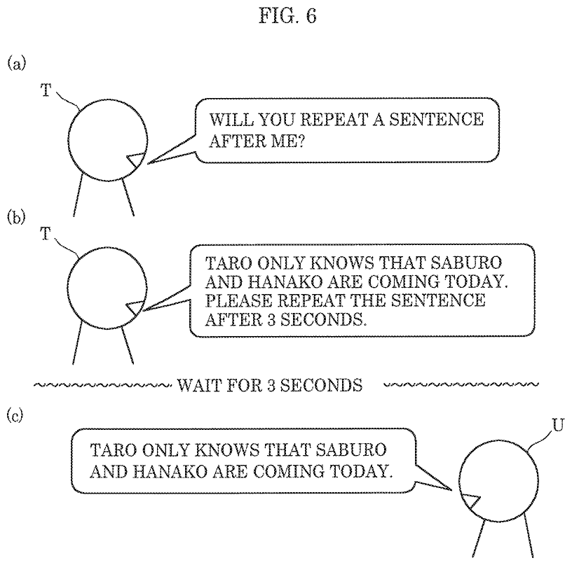

FIG. 6 is a diagram showing a second example of a method for acquiring speech data. As in the example of FIG. 5, in the example of FIG. 6 as well, evaluatee U is given a task to verbally repeat a sentence spoken by tester T. Tester T first gives an instruction to verbally repeat a sentence to evaluatee U ((a) in FIG. 6), and then speaks a sentence ((b) in FIG. 6). At this time, an instruction to start speaking after a predetermined time (for example, after 3 seconds) is also given to evaluatee U. That is, in the example shown in FIG. 6, evaluatee U is given a task to verbally repeat a sentence uttered by tester T after a predetermined time. Then, evaluatee U waits for the predetermined time to elapse, and speaks in response to the task ((c) in FIG. 6). Acquirer 110 acquires speech data of the speech uttered by evaluatee U. The waiting time during which evaluatee U waits to speak is used for the purpose of applying a certain level of stress to the brain of evaluatee U.

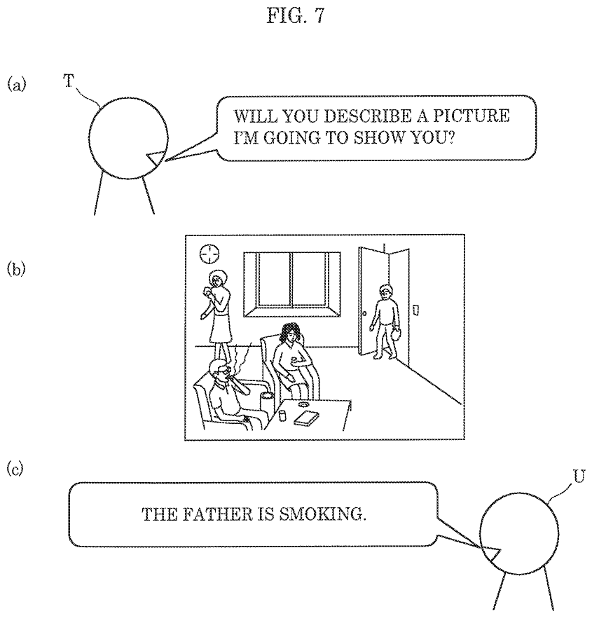

FIG. 7 is a diagram showing a third example of a method for acquiring speech data. In the example shown in FIG. 7, a task to verbally describe a picture or a photograph presented by tester T is given to evaluatee U. Tester T first gives an instruction to verbally describe a picture or a photograph to evaluatee U ((a) in FIG. 7), and presents the picture or the photograph ((b) in FIG. 7). Evaluatee U verbally describes the picture or the photograph presented to evaluatee U by verbally listing as many things as possible ((c) in FIG. 7). Acquirer 110 acquires speech data of the speech uttered by evaluatee U.

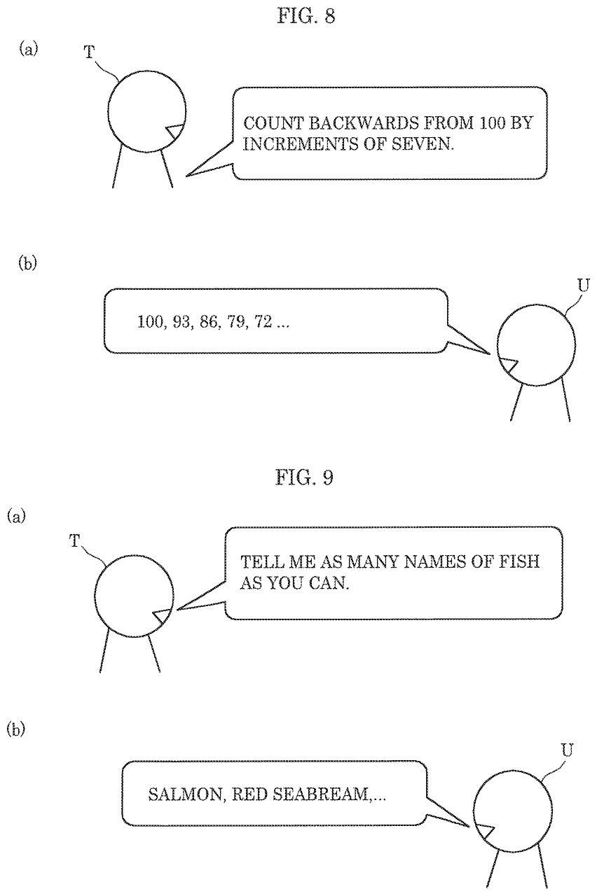

FIG. 8 is a diagram showing a fourth example of a method for acquiring speech data. In the example shown in FIG. 8, a task to count numbers aloud based on a condition specified by tester T is given to evaluatee U. Tester T first specifies a condition ((a) in FIG. 8), and evaluatee U counts numbers aloud based on the condition given to evaluatee U ((b) in FIG. 8). Acquirer 110 acquires speech data of the speech uttered by evaluatee U.

FIG. 9 is a diagram showing a fifth example of a method for acquiring speech data. In the example shown in FIG. 9, a task to verbally list words that satisfy a condition specified by tester T is given to evaluatee U. Tester T first specifies a condition ((a) in FIG. 9), and evaluatee U verbally lists words that satisfy the condition given to evaluatee U ((b) in FIG. 9). Acquirer 110 acquires speech data of the speech uttered by evaluatee U. The condition specified by tester T is, for example, category (the name of fish or the like), but may be nouns beginning with the letter “e”, or the like.

### [Feature Value: Proportion of Speaking Time]

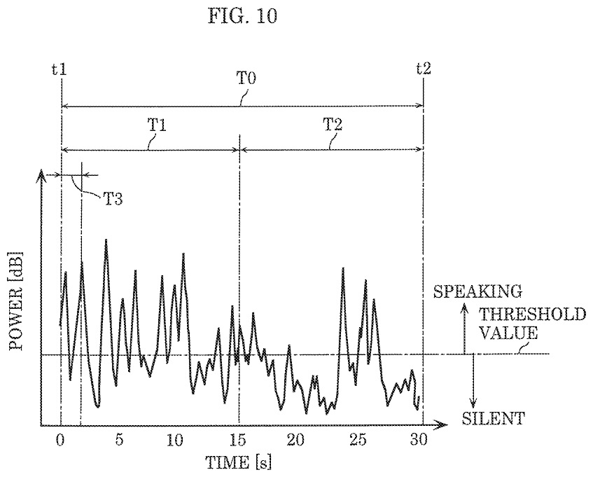

Next, the feature value calculated in step S102 will be described. Calculator 120 calculates, for example, the proportion of speaking time of evaluatee U as the feature value. FIG. 10 is a diagram illustrating the proportion of speaking time.

FIG. 10 shows variations in the power of speech data that indicates speech uttered by evaluatee U with time. The speech data includes: a speaking period during which evaluatee U is uttering speech; and a non-speaking period during which evaluatee U is silent. Calculator 120 determines, for example, a period during which the power is greater than or equal to a predetermined threshold value (for example, −50 dB) as the speaking period during which evaluatee U is uttering speech. Also, calculator 120 determines a period during which the power is less than the predetermined threshold value as the non-speaking period during which evaluatee U is silent.

The speaking time is the total period of time during which evaluatee U is uttering speech. Calculator 120 calculates the proportion of speaking time in a predetermined period within the speech data acquired by acquirer 110. Here, the predetermined period is, for example, a period between timing t1 at which tester T has given a task and timing t2 at which evaluatee U has finished responding to the task (that is, the total period until the end of the test being represented by T0). Timing t1 and timing t2 may be specified through voice recognition or the like, or may be designated by a person who performs evaluation such as tester T via a user interface (not shown) included in cognitive function evaluation device 100. Timing t2 may be set in advance to, for example, 30 seconds after timing t.

Evaluatee U who takes pauses while speaking is considered to have cognitive function impairment. Also, in the case where a task as shown in FIG. 7 or 9 to verbally list things is given to evaluatee U, if evaluatee U has cognitive function impairment, the number of things evaluatee U can verbally list is small, and thus the speaking time is shortened. Accordingly, evaluator 130 determines that the level of cognitive impairment is lower as the proportion of speaking time is lower.

The predetermined period described above is not limited to total period T0 until the end of the test. For example, the predetermined period may be a period that belongs to the latter half of speech data (or in other words, the latter half of total period T0). For example, when a task as shown in FIG. 9 to verbally list things is given to evaluatee U, irrespective of the level of cognitive function of evaluatee U, the number of words evaluatee U can think of decreases gradually, and the speaking time is shortened. However, evaluatee U who has a lower level of cognitive function impairment can think of only a smaller number of words, and it is therefore expected that the speaking time ends at an early stage. Accordingly, the proportion of speaking time in the predetermined period that belongs to the latter half of speech data may vary significantly according to the level of cognitive function, and thus is suitable for use in cognitive function evaluation.

Also, calculator 120 may calculate a variation in the proportion of speaking time. For example, as shown in FIG. 10, in the case where the total period until the end of the test is divided into two periods: first period T1 and second period T2, calculator 120 may calculate the difference between the proportion of speaking time in first period T1 and the proportion of speaking time in second period T2 (that is, the amount of variation). Calculator 120 may calculate the ratio between the proportion of speaking time in first period T1 and the proportion of speaking time in second period T2 (that is, the rate of variation). In either case, evaluator 130 evaluates cognitive function based on comparison between the proportion of speaking time in first period T1 and the proportion of speaking time in second period T2.

As described above, it is considered that as evaluatee U has a lower level of cognitive function impairment, the speaking time in the latter half of total period T0 is shorter. It is therefore considered that, as evaluatee U has a lower level of cognitive function impairment, the variation in the proportion of speaking time (relative to the proportion of speaking time at first period T1) is larger. Accordingly, evaluator 130 determines that the level of cognitive impairment is lower as the variation in the proportion of speaking time is larger. Total period T0 may be divided into three periods or more.

### [Feature Value: Total Period, Time Until Evaluatee Starts Uttering Speech]

Calculator 120 may calculate total period T0 shown in FIG. 10 as the feature value. That is, calculator 120 may calculate the time from when a task is given to evaluatee U to when evaluatee U finishes responding to the task as the feature value.

For example, in the case where a task as shown in FIG. 5 or 6 to verbally repeat a sentence is given to evaluatee U, if evaluatee U has a lower level of cognitive function impairment, it is difficult for evaluatee U to verbally repeat a sentence due to memory loss, and it is therefore expected that it takes time for evaluatee U to finish verbally repeating a sentence. Accordingly, evaluator 130 determines that the level of cognitive impairment is lower as total period T0 is longer.

Also, for example, in the case where a task as shown in FIG. 7 or 9 to verbally list things is given to evaluatee U, if evaluatee U has a lower level of cognitive function impairment, it is expected that the speaking time ends at an early stage because the number of things evaluatee U can verbally list is small. Accordingly, in this case, evaluator 130 determines that the level of cognitive impairment is lower as total period T0 is shorter.

Also, calculator 120 may calculate time T3 from when a task is given to evaluatee U to when evaluatee U starts uttering speech (see FIG. 10) as the feature value. If evaluatee U has a lower level of cognitive function impairment, the reaction speed is low, and thus it is expected that it takes time for evaluatee U to start uttering speech after the task is given. Accordingly, evaluator 130 determines that the level of cognitive impairment is lower as time T3 is longer.

### [Feature Value: Fundamental Frequency]

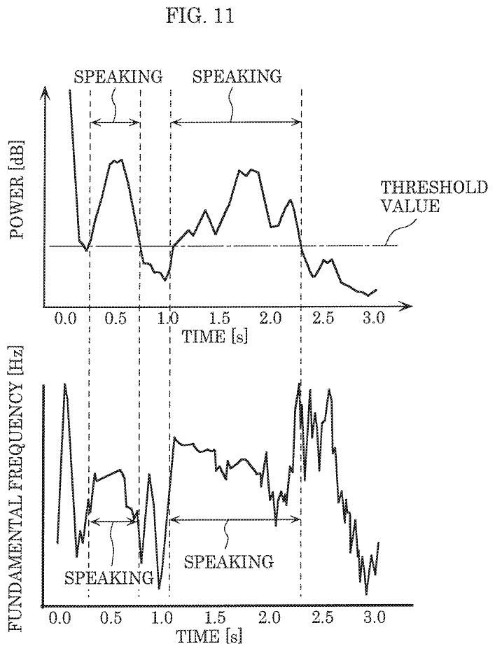

Calculator 120 may calculate a variation per unit time in the fundamental frequency in the speech data as the feature value. FIG. 11 is a diagram showing variations in fundamental frequency with time.

Fundamental frequency is the number of oscillations of the source of sound, or in other words, it means the pitch of the speech uttered by evaluatee U. As used herein, the term “variation per unit time in the fundamental frequency” means a differential value of the fundamental frequency. The fundamental frequency is calculated from, for example, the first peak of autocorrelation function, but may be calculated by using any other methods.

If evaluatee U has a lower level of cognitive function impairment, evaluatee U shows less emotional responses and speaks with a flat and toneless voice. It is therefore expected that the variation in the fundamental frequency is small. In addition, if evaluatee U has a lower level of cognitive function impairment, due to a reduction in the physical function, evaluatee U may not sufficiently breathe (or may not sufficiently use the lungs) when uttering speech, and it is therefore expected that the variation in the fundamental frequency is small. Accordingly, evaluator 130 determines that the level of cognitive impairment is lower as, for example, the variation per unit time in the fundamental frequency in the speech data is smaller.

Also, calculator 120 may calculate the variation range per unit time in the fundamental frequency in the speech data as the feature value. As used herein, the term “the variation range per unit time in the fundamental frequency” means a difference between the maximum value and the minimum value per unit time in the fundamental frequency. In this case, for the same reason as that described for the variation in the fundamental frequency, evaluator 130 determines that the level of cognitive impairment is lower as the variation range per unit time in the fundamental frequency in the speech data is smaller.

In the case where calculator 120 calculates the variation in the fundamental frequency or the variation range in the fundamental frequency, among the speech data, only the speaking period during which evaluatee U is uttering speech is used in the calculation. As shown in FIG. 11, calculator 120 can determine a period during which the power is greater than or equal to a threshold value as the speaking period during which evaluatee U is uttering speech. As a result of the speaking period during which evaluatee U is uttering speech being selectively used in the calculation, the accuracy of evaluation performed by evaluator 130 is improved.

Also, calculator 120 may calculate, as the feature value, the difference or ratio between a variation (or a variation range) in the fundamental frequency in the first predetermined period (for example, 5 seconds) in the speaking period during which evaluatee U is uttering speech and a variation (or a variation range) in the fundamental frequency in the last predetermined period (for example, 5 seconds) in the speaking period during which evaluatee U is uttering speech. For example, calculator 120 may use a physical quantity obtained by normalizing the variation (or the variation range) in the fundamental frequency in the last predetermined period with the variation (or the variation range) in the fundamental frequency in the first predetermined period, as the feature value.

### [Evaluation Method]

Evaluator 130 evaluates the cognitive function of evaluatee U by using at least one of the feature values described above. However, the accuracy of evaluation increases if evaluation is performed collectively by using a plurality of feature values. For example, evaluator 130 may evaluate the cognitive function of evaluatee U by performing a first evaluation based on the proportion of speaking time of evaluatee U in a predetermined period in the speech data and a second evaluation based on the fundamental frequency in the speech data. For example, evaluator 130 can evaluate the cognitive function of evaluatee U by using a plurality of feature values by adding together a score in the first evaluation determined based on reference data 151 and a score in the second evaluation determined based on reference data 151. When adding the scores together, the scores may be weighted according to the feature value.

Reference data 151 is, for example, data that indicates the correspondence relationship between scores in MoCA test and feature values. It is sufficient that reference data 151 is data that can be compared with the feature values so as to evaluate the level of cognitive function, or data that indicates the relationship between scores such as scores in MMSE (Mini-Mental State Examination) and feature values.

Also, in the case where evaluation is performed by determining whether or not there is a sign of dementia based on each feature value, evaluator 130, if, for example, all of a plurality of feature values used in the evaluation show that there is a sign of dementia, it is determined that evaluatee U shows a sign of dementia. In other words, if at least one of a plurality of feature values used in the evaluation shows that there is no sign of dementia, evaluator 130 determines that evaluatee U shows no sign of dementia. However, evaluator 130 may determine that evaluatee U shows a sign of dementia if at least one of a plurality of feature values used in the evaluation shows that there is a sign of dementia. In other words, evaluator 130 may determine that evaluatee U shows no sign of dementia if all of a plurality of feature values used in the evaluation show that there is no sign of dementia.

### Advantageous Effects, Etc

As described above, cognitive function evaluation device 100 includes: acquirer 110 that acquires speech data that indicates speech uttered by evaluatee U in response to a task given to evaluatee U; evaluator 130 that evaluates a cognitive function of evaluatee U by performing a first evaluation based on a proportion of speaking time of evaluatee U in a predetermined period in the speech data acquired by acquirer 110 and a second evaluation based on a fundamental frequency in the speech data acquired by acquirer 110; and outputter 140 that outputs an evaluation result obtained from the evaluation performed by evaluator 130.

As a result of cognitive function evaluation device 100 described above acquiring speech data, the cognitive function of evaluatee U can be easily evaluated. Also, two feature values including speaking time and fundamental frequency are used, and thus the accuracy of evaluation is improved.

Also, for example, evaluator 130 performs the first evaluation based on the proportion of speaking time of evaluatee U in the predetermined period that belongs to a latter half of the speech data.

It is expected that the difference in speaking time is likely to be pronounced in the latter half of the speech data due to the cognitive function. Accordingly, by using cognitive function evaluation device 100 configured as described above, the accuracy of evaluation of the cognitive function of evaluatee U can be improved.

Also, for example, evaluator 130 may perform the first evaluation based on a comparison between the proportion of speaking time of evaluatee U in a first period in the speech data and the proportion of speaking time of evaluatee U in a second period in the speech data, the second period being after the first period.

Cognitive function evaluation device 100 configured as described above can evaluate the cognitive function of evaluatee U based on a variation in the proportion of speaking time with time.

Also, for example, evaluator 130 may perform the second evaluation based on a variation per unit time in the fundamental frequency.

Cognitive function evaluation device 100 configured as described above can evaluate the cognitive function of evaluatee U based on the variation per unit time in the fundamental frequency in the speech data.

Also, for example, evaluator 130 may perform the second evaluation based on a variation range per unit time in the fundamental frequency.

Cognitive function evaluation device 100 configured as described above can evaluate the cognitive function of evaluatee U based on the variation range per unit time in the fundamental frequency in the speech data.

Also, for example, evaluator 130 evaluates the cognitive function of evaluatee U by further performing a third evaluation based on a time from when the task is given to evaluatee U and to when evaluatee U starts uttering speech.

Cognitive function evaluation device 100 configured as described above can evaluate the cognitive function of evaluatee U based on the time from when the task is given to evaluatee U and to when evaluatee U starts uttering speech.

Also, for example, evaluator 130 may evaluate the cognitive function of evaluatee U by further performing a third evaluation based on a time from when the task is given to evaluatee U to when evaluatee U finishes responding to the task.

Cognitive function evaluation device 100 configured as described above can evaluate the cognitive function of evaluatee U based on the time from when the task is given to evaluatee U to when evaluatee U finishes responding to the task.

Also, for example, the speech data indicates speech uttered by evaluatee U in response to the task that is a task to verbally repeat a sentence spoken by tester T.

Cognitive function evaluation device 100 configured as described above can use, as data to be processed, the speech data that indicates the speech uttered by evaluatee U in response to the task to verbally repeat a sentence spoken by tester T.

Also, for example, the sentence is a sentence that contains 5 phrases or more that include a plurality of pairs of a subject and a predicate.

With this configuration, it is possible to cause the level of dementia of evaluatee U to be significantly noticeable in the speech data. Also, acquirer 110 can acquire speech data for a relatively long time.

Also, for example, the speech data indicates speech uttered by evaluatee U in response to the task that is a task to verbally repeat a sentence spoken by tester T after a predetermined time.

Cognitive function evaluation device 100 configured as described above can use, as data to be processed, the speech data that indicates the speech uttered by evaluatee U in response to the task to verbally repeat a sentence spoken by tester T after a predetermined time.

Also, for example, the speech data indicates speech uttered by evaluatee U in response to the task that is a task to verbally describe a picture or a photograph presented by tester T.

Cognitive function evaluation device 100 configured as described above can use, as data to be processed, the speech data of the speech uttered by evaluatee U in response to the task to verbally describe a picture or a photograph presented by tester T.

Also, for example, the speech data indicates speech uttered by evaluatee U in response to the task that is a task to count numbers aloud based on a condition specified by tester T.

Cognitive function evaluation device 100 configured as described above can use, as data to be processed, the speech data that indicates the speech uttered by evaluatee U in response to the task to count numbers aloud based on the condition specified by tester T.

Also, for example, the speech data indicates speech uttered by evaluatee U in response to the task that is a task to verbally list words that satisfy a condition specified by tester T.

Cognitive function evaluation device 100 configured as described above can use, as data to be processed, the speech data that indicates the speech uttered by evaluatee U in response to the task to verbally list words that satisfy the condition specified by tester T.

Also, cognitive function evaluation system 200 includes cognitive function evaluation device 100, sound collecting device 300 that detects the speech uttered by evaluatee U, and display device 400 that displays an evaluation result output by outputter 140.

Cognitive function evaluation system 200 configured as described above can easily evaluate the cognitive function of evaluatee U by acquiring speech data. Also, two feature values including speaking time and fundamental frequency are used, and thus the accuracy of evaluation is improved.

Also, a cognitive function evaluation method according to an embodiment is a cognitive function evaluation method executed by a computer. The cognitive function evaluation method includes: acquiring speech data that indicates speech uttered by evaluatee U in response to a task given to evaluatee U; evaluating a cognitive function of evaluatee U by performing a first evaluation based on a proportion of speaking time of evaluatee U in a predetermined period in the speech data acquired in the acquiring of the speech data and a second evaluation based on a fundamental frequency in the speech data acquired in the acquiring of the speech data; and outputting an evaluation result obtained from the evaluation in the evaluating of the cognitive function of evaluatee U.

With the cognitive function evaluation method, the cognitive function of evaluatee U can be easily evaluated by acquiring the speech data. Also, two feature values including speaking time and fundamental frequency are used, and thus the accuracy of evaluation is improved.

Also, the present invention may be implemented as a program for causing a computer to execute the cognitive function evaluation method described above.

With the program, the cognitive function of evaluatee U can be easily evaluated. Also, two feature values including speaking time and fundamental frequency are used, and thus the accuracy of evaluation is improved.

### Variations

Next, cognitive function evaluation systems according to Variations 1 and 2 of the embodiment will be described. Structural elements that are the same as those of the embodiment are given the same reference numerals, and a redundant description may be omitted or simplified.

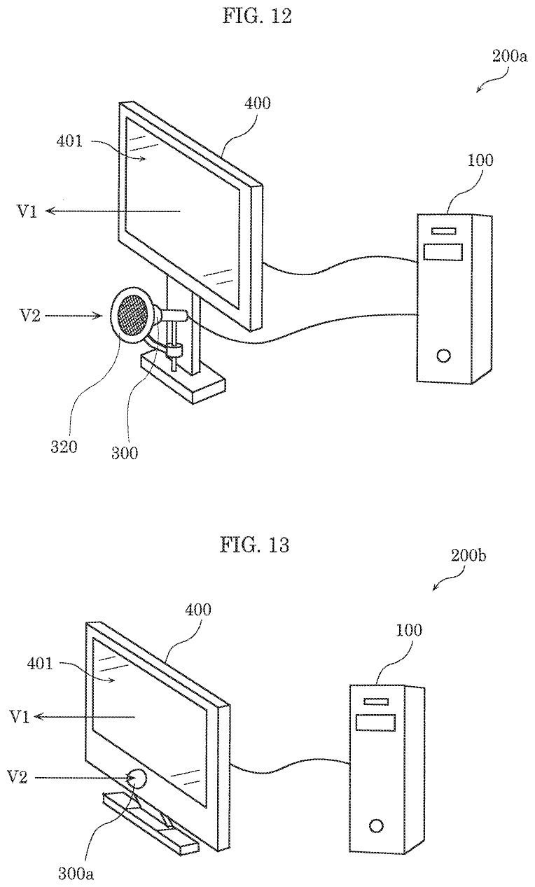

FIG. 12 is a diagram showing a configuration of a cognitive function evaluation system according to Variation 1 of the embodiment.

Cognitive function evaluation system 200a according to Variation 1 of the embodiment includes, as with cognitive function evaluation system 200 of the embodiment, cognitive function evaluation device 100, sound collecting device 300, and display device 400. Cognitive function evaluation system 200a may include, for example, pop filter 320 so as to cover sound collecting device 300.

Also, in cognitive function evaluation system 200a, sound collecting device 300 that has directivity is used. Here, sound collecting device 300 and display device 400 are disposed such that a direction in which sound collecting device 300 exhibits a maximum sensitivity (sound collecting direction V2 shown in FIG. 12) matches normal direction V1 of display surface 401 of display device 400 where task information is displayed. Specifically, sound collecting device 300 and display device 400 are disposed on a fixture such as a desk such that normal direction V1 and sound collecting direction V2 are parallel to each other. Sound collecting device 300 and display device 400 may be fixed to a structural member or the like. Also, cognitive function evaluation system 200a may include a fixing item for fixing the positional relationship between sound collecting device 300 and display device 400.

With this configuration, even when evaluatee U speaks while looking at display device 400, sound collecting direction V2 easily matches the direction in which evaluatee U speaks. For this reason, by disposing sound collecting device 300 and display device 400 in the positional relationship as described above in cognitive function evaluation system 200a, sound collecting device 300 can accurately detect the speech uttered by evaluatee U.

Next, a cognitive function evaluation system according to Variation 2 of the embodiment will be described.

FIG. 13 is a diagram showing a configuration of a cognitive function evaluation system according to Variation 2 of the embodiment.

Cognitive function evaluation system 200b according to Variation 2 of the embodiment includes, as with cognitive function evaluation system 200 of the embodiment, cognitive function evaluation device 100, sound collecting device 300a, and display device 400.

Sound collecting device 300a is a microphone that detects speech uttered by evaluatee U and outputs speech data that indicates the detected speech to cognitive function evaluation device 100, as with sound collecting device 300. Also, sound collecting device 300a has directivity as with sound collecting device 300 included in cognitive function evaluation system 200a according to Variation 1 of the embodiment.

In cognitive function evaluation system 200b, sound collecting device 300a and display device 400 are unitarily formed as a single structural element. Specifically, sound collecting device 300a and display device 400 are disposed in the same housing. By unitarily forming sound collecting device 300a and display device 400 as a single structural element such that normal direction V1 and sound collecting direction V2 match with each other during the production process, a situation is suppressed in which normal direction V1 and sound collecting direction V2 are offset from each other when cognitive function evaluation system 200b is used by evaluatee U.

### Other Embodiments

The cognitive function evaluation systems and the like according to the embodiment and Variations 1 and 2 of the embodiment have been described above, but the present invention is not limited to the embodiment given above.

For example, in the embodiment given above, a task is given to the evaluatee to acquire speech data, but giving a task is not a requirement. It is sufficient that the acquirer acquires speech data that indicates speech uttered by the evaluatee.

For example, in the embodiment given above, Alzheimer's disease is given as a specific example of a symptom of cognitive function impairment. Here, the term “cognitive function” refers to the abilities of recognition, remembering information, making decisions, and the like, and the term “dementia” refers to symptoms of impairment of the cognitive function. That is, the cognitive function evaluated by the cognitive function evaluation device is not limited to Alzheimer's disease, and may be, for example, vascular dementia or the like.

Also, in the embodiment given above, a task is given by the tester. However, a task may be given by the cognitive function evaluation device via the display device or the like.

Also, the present invention may be implemented as a program for causing a computer to execute the steps performed by the cognitive function evaluation device. Also, the present invention may be implemented as a computer readable recording medium, such as a CD-ROM, on which the program is recorded. Also, the present invention may be implemented as information, data or a signal that indicates the program. The program, the information, the data, and the signal may be distributed via a communication network such as the Internet.

The present invention also encompasses other embodiments obtained by making various modifications that can be conceived by a person having ordinary skill in the art to the above embodiment as well as embodiments implemented by any combination of the structural elements and the functions of the above embodiment without departing from the scope of the present invention.

### REFERENCE MARKS IN THE DRAWINGS

100 cognitive function evaluation device
110 acquirer
130 evaluator
140 outputter
200, 200a, 200b cognitive function evaluation system
300, 300a sound collecting device
400 display device
T tester
T1 first period
T2 second period
U evaluatee

1. A cognitive function evaluation device, comprising:
an acquirer that acquires speech data that indicates speech uttered by an evaluatee in response to a task given to the evaluatee;
an evaluator that evaluates a cognitive function of the evaluatee by performing a first evaluation based on a proportion of speaking time of the evaluatee in a predetermined period in the speech data acquired by the acquirer and a second evaluation based on a fundamental frequency in the speech data acquired by the acquirer; and
an outputter that outputs an evaluation result obtained from the evaluation performed by the evaluator.

2. The cognitive function evaluation device according to claim 1,
wherein the evaluator performs the first evaluation based on the proportion of speaking time of the evaluatee in the predetermined period that belongs to a latter half of the speech data.

3. The cognitive function evaluation device according to claim 1,
wherein the evaluator performs the first evaluation based on a comparison between the proportion of speaking time of the evaluatee in a first period in the speech data and the proportion of speaking time of the evaluatee in a second period in the speech data, the second period being after the first period.

4. The cognitive function evaluation device according to claim 1,
wherein the evaluator performs the second evaluation based on a variation per unit time in the fundamental frequency.

5. The cognitive function evaluation device according to claim 1,
wherein the evaluator performs the second evaluation based on a variation range per unit time in the fundamental frequency.

6. The cognitive function evaluation device according to claim 1,
wherein the evaluator evaluates the cognitive function of the evaluatee by further performing a third evaluation based on a time from when the task is given to the evaluatee to when the evaluatee starts uttering speech.

7. The cognitive function evaluation device according to claim 1,
wherein the evaluator evaluates the cognitive function of the evaluatee by further performing a third evaluation based on a time from when the task is given to the evaluatee to when the evaluatee finishes responding to the task.

8. The cognitive function evaluation device according to claim 1,
wherein the speech data indicates the speech uttered by the evaluatee in response to the task that is a task to verbally repeat a sentence spoken by a tester.

9. The cognitive function evaluation device according to claim 8,
wherein the sentence is a sentence that contains 5 phrases or more that include a plurality of pairs of a subject and a predicate.

10. The cognitive function evaluation device according to claim 8,
wherein the speech data indicates the speech uttered by the evaluatee in response to the task that is a task to verbally repeat, after a predetermined time, a sentence spoken by the tester.

11. The cognitive function evaluation device according to claim 1,
wherein the speech data indicates the speech uttered by the evaluatee in response to the task that is a task to verbally describe a picture or a photograph presented by a tester.

12. The cognitive function evaluation device according to claim 1,
wherein the speech data indicates the speech uttered by the evaluatee in response to the task that is a task to count numbers aloud based on a condition specified by a tester.

13. The cognitive function evaluation device according to claim 1,
wherein the speech data indicates the speech uttered by the evaluatee in response to the task that is a task to verbally list words that satisfy a condition specified by a tester.

14. A cognitive function evaluation system, comprising:
the cognitive function evaluation device according to claim 1;
a sound collecting device that detects speech uttered by the evaluatee; and
a display device that displays an evaluation result output by the outputter.

15. A cognitive function evaluation method executed by a computer, the cognitive function evaluation method comprising:
acquiring speech data that indicates speech uttered by an evaluatee in response to a task given to the evaluatee;
evaluating a cognitive function of the evaluatee by performing a first evaluation based on a proportion of speaking time of the evaluatee in a predetermined period in the speech data acquired in the acquiring of the speech data and a second evaluation based on a fundamental frequency in the speech data acquired in the acquiring of the speech data; and
outputting an evaluation result obtained from the evaluation in the evaluating of the cognitive function of the evaluatee.

16. A non-transitory computer-readable recording medium for use in a computer, the recording medium having a computer program recorded thereon that causes a computer to execute the cognitive function evaluation method according to claim 15.
